(19) United States
(12) Patent Application Publication
(10) Pub. No.: US 2007/0038299 A1
(43) Pub. Date: Feb. 15, 2007

(54) Multilayer microperforated implant

(75) Inventors: Kevin T. Stone, Winona Lake, IN (US); Karen Troxel, Warsaw, IN (US)

Correspondence Address: HARNESS, DICKEY & PIERCE, P.L.C., P.O. BOX 828, BLOOMFIELD HILLS, MI, 48303, US

(73) Assignee: Arthrotek, Inc, Warsaw, IN (US)

(21) Appl. No.: 11/203,643

(22) Filed: Aug. 12, 2005

(51) Int. Cl. A61F 2/02

(52) U.S. Cl. 623/11.11; 623/23.75

(57) ABSTRACT

A multilayer microperforated implant comprising a plurality of microperforated substrate layers is provided. Methods of forming a microperforated implant comprise providing at least one substrate layer; perforating at least a region of the substrate layer; and dehydrating the substrate layer. Methods of augmenting a site in need of repair with the microperforated implant are also provided.

### FIELD

The present invention relates to multilayer microperforated implants.

### BACKGROUND

Soft tissue implants may be advantageously made of resorbable materials. The resorbable materials facilitate tissue growth into the implant because as the material resorbs, new tissue fills the voids caused by resorption without compromising the strength of the implant area. Soft tissue implants may be single layer implants or multilayer implants. The multilayer tissue implants combine several layers or sheets of a substrate to provide enhanced strength and allow tailoring of the implant for specific applications.

Resorbable materials and some partially or non-resorbable materials are sensitive to moisture and are shipped dehydrated to prevent premature degradation of the implant. Hydration of a dehydrated single layer or multilayer implant requires time, but rehydration of multilayer implants is an especially cumbersome process because of the distances traveled by a hydration fluid. Hydration of the inner regions of each layer takes place from the exposed edges. The hydration liquid travels from the exposed edges to the center of the layer, and the top and bottom of each layer must be hydrated. This is repeated for each layer until all exposed edges and innermost regions and layers are hydrated. The rehydration process generally includes soaking the dehydrated multilayer implant in a hydration liquid for several hours, agitating the implant in the hydration liquid, and using a large quantity of the hydration liquid.

Although instructions provided with the dehydrated implants may detail hydration for several hours, it may be medically necessary to rehydrate the implant in a shorter amount of time. Improperly following instructions may result in an incomplete rehydration or with a multilayer implant, an incomplete rehydration where only the outermost layers or the perimeter of the implant is in proper condition for use. Incomplete hydration may hinder integration of newly formed tissues into the partially hydrated layers.

It may be desirable to provide an implant which promotes soft tissue ingrowth, hydrates rapidly, promotes new tissue ingrowth, and has high user compliance.

### SUMMARY OF THE INVENTION

Various embodiments of the present provide a multilayer microperforated implant comprising a plurality of microperforated substrate layers. The microperforations have a diameter of less than about 10 micrometers. The implant may have at least one substrate layer having a punch density of from about 1 to about 1,000 punches per square inch. The substrate layers may be made of resorbable materials such as polysaccharides, synthetic polymers, natural polymers, and mixtures thereof. Polysaccharides may include hyaluronic acid, chitin, chitosan, alginate, carboxymethylcellulose, and mixtures thereof. Synthetic polymers may include polymers and co-polymers of glycolic acid, L-lactic acid, D-lactic acid, urethane urea, trimethylene carbonate, dioxanone, caprolactone, hydroxybutyrate, orthoesters, orthocarbonates, aminocarbonates, and physical combinations thereof. Natural polymers may include collagen, elastin, silk, fibrin, fibrinogen, and mixtures thereof. The collagen may be porcine derived. The implant may include at least 8 layers and may be dehydrated. At least one microperforation in each substrate layer may be in fluid communication with at least one microperforation in an adjacent substrate layer. The microperforations may be arranged to direct a hydration media to the innermost substrate layers. The implant may be pre-fabricated and may be used as a cartilage, tendon, or ligament implant.

Various embodiments of the present invention also provide methods of forming a microperforated implant comprising: providing at least one substrate layer; perforating at least a region of the substrate layer; and dehydrating the substrate layer. A plurality of substrate layers may also be employed and the plurality of layers is stacked to form a multilayer implant. Perforating the layer may be achieved by contacting the substrate layer with a needle to displace less than about 10 micrometers of the material and form an opening. The opening may be stretched to a greater diameter without removing any substrate material. The dehydrated microperforated implant may be contacted with a hydration fluid to rehydrate the implant as the fluid transverses each of the substrate layers. Hydration fluids may include water, saline, and blood such as whole blood and platelet concentrate.

Various embodiments of the present invention also provide methods of augmenting a site in need of repair, comprising: providing a multilayer implant comprising a plurality of substrate layers, wherein the substrate layers include perforations having a diameter of less than about 10 micrometers; and placing the implant at a site in need of soft tissue repair. The implant may be provided in a dehydrated state or may be preformed into a shape. Hydration media may include water, saline, and blood.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 depicts a side view of a multilayer implant according to various embodiments;

FIG. 2 depicts a side view of a multilayer implant according to various embodiments;

FIG. 3 depicts an enlarged side view of a layer of an implant according to various embodiments;

FIG. 4 depicts an enlarged side view of a layer of an implant according to various embodiments;

FIG. 5 is a flow chart illustrating a method of forming an implant according to various embodiments;

FIG. 6 depicts a rolling device used to form an implant according to various embodiments; and

FIG. 7 depicts a plating device used to form an implant according to various embodiments.

### DETAILED DESCRIPTION OF THE PREFERRED EMBODIMENTS

The following description of the preferred embodiment(s) is merely exemplary in nature and is in no way intended to limit the invention, its application, or uses. Although various embodiments may be described in conjunction with a collagen substrate or for use with a shoulder, elbow, or finger, it is understood that the microperforated implants and methods of the invention may be of any appropriate substrate or shape and may be used with any appropriate procedure and not solely those illustrated.

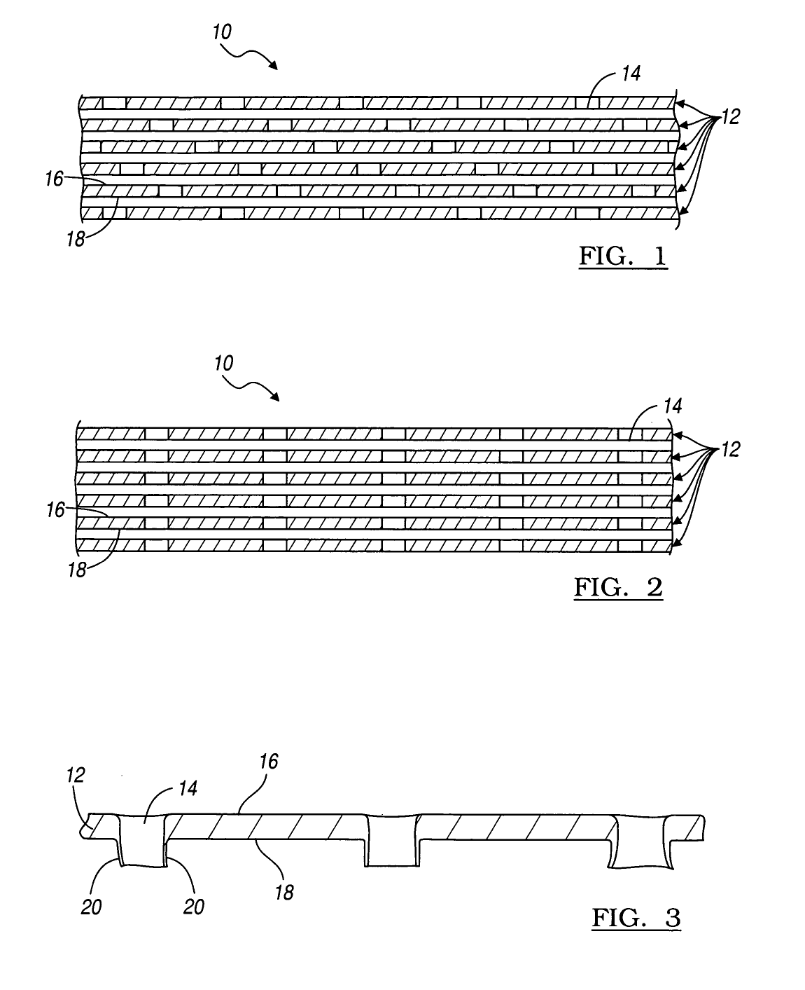

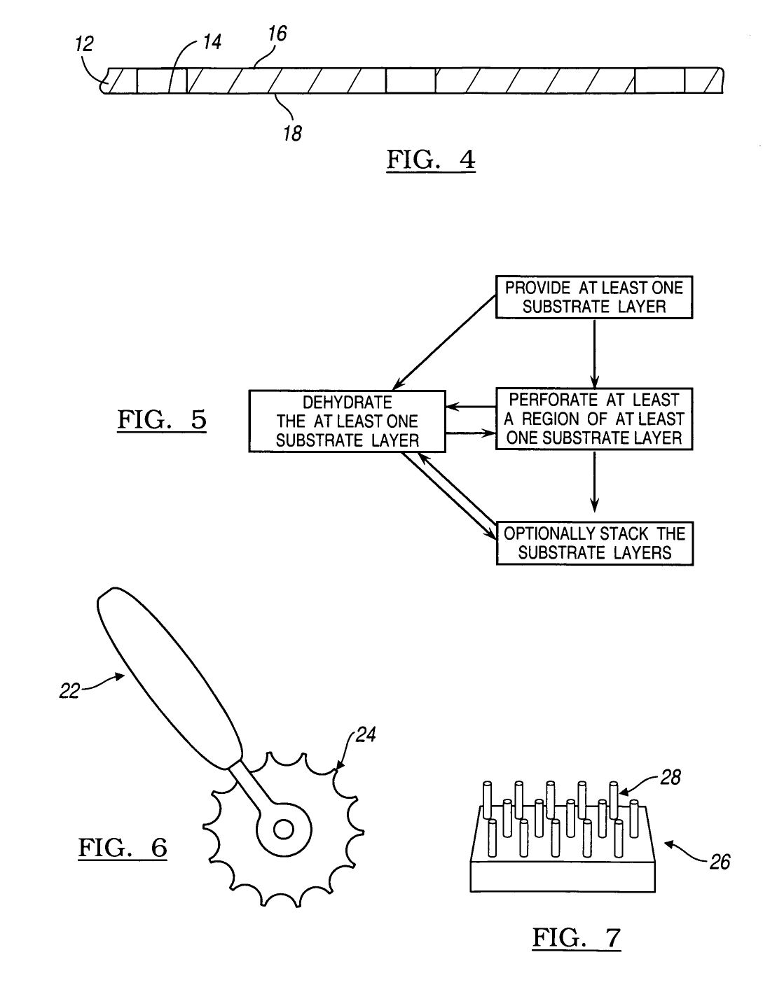

Referring to FIGS. 1 through 4, the multilayer implant 10 comprises a plurality of substrate layers 12 having microperforations 14. The multilayer implant 10 material may be dehydrated to a final moisture content of less than about 5% using techniques known in the art including oven drying, air drying, vacuum drying, or freeze drying.

The substrate layers 12 include a top surface 16 and a bottom surface 18. The substrate layers 12 may be made of bioresorbable synthetic polymers, natural polymers, polysaccharides, and mixtures thereof. Synthetic bioresorbable materials may include, but are not limited to, polymers and copolymers of glycolic acid, L-lactic acid, D-lactic acid, urethane urea, trimethylene carbonate, dioxanone, caprolactone, hydroxybutyrate, orthoesters, orthocarbonates, aminocarbonates, and physical combinations thereof. Other polymerizable hydroxy acids may also be employed. Natural polymers may include collagen, elastin, silk, fibrin, fibrinogen, other naturally occurring tissue-derived proteins, and mixtures thereof. Natural polysaccharides may include, without limitation, hyaluronic acid, chitin, chitosan, alginate, carboxymethylcellulose, other polysaccharides, and mixtures thereof. The substrate layers 12 may also be non-resorbable materials from any suitable source, including resorbable materials such as those listed above that have been treated to become non-resorbable.

In preferred embodiments, the substrate layers 12 may be collagen. The membranous collagen may be naturally derived from tissue such as submucosal intestine, or may be fabricated by casting a collagen solution into a membrane. The collagen substrate may be from a xenograft source, an allograft source, or a synthetic source (e.g., a collagen not derived from an animal or plant source and manufactured, such as in a laboratory). For example, a porcine collagen may be used for at least one collagen substrate layer. Porcine collagen is readily available, provides flexibility of the collagen substrate, and is durable. Depending on the end use of the multilayer implant 10, the collagen substrate layer 12 may be from any collagen source (e.g. human, porcine, or bovine) which provides the desired durability, flexibility, and resorbability or permanence.

The collagen substrate layer 12 may uncrosslinked (0% linkages), partially crosslinked (greater than 0% and less than 100% linkages), or fully crosslinked (100% linkages). The collagen substrate layer 12 may be sufficiently crosslinked to be non-immunogenic while also being resorbable. In various embodiments, the sufficient crosslinking to achieve non-immunogenic and resorbable substrate layer 12 may be from about 10% to about 90% linkages, from about 30% to about 70% linkages, or from about 40% to about 60% linkages. One skilled in the art appreciates that the resorption rate of the collagen substrate layer 12, and accordingly a collagen based multilayer implant 10, increases with the amount of crosslinked bonds. Selection of the amount of crosslinking depends on the desired longevity of the implant 10. For example, in a highly crosslinked collagen substrate layer 12 having 85% crosslinked bonds, the collagen substrate layer 12 may remain implanted and substantially intact inside of a recipient for months, decades, or a lifetime. A sufficient amount of crosslinking may ensure that the collagen substrate layer 12 does not degrade, deform, or otherwise lose strength too rapidly over the life of the implant. In contrast, a lesser crosslinked collagen substrate layer 12 having about 10% linkage, may be for temporary use and designed to retain the majority of its structural integrity for only a few days, weeks, or months. This may be useful in less load bearing areas of the body or in situations where the repair is minor and may be replaced with regenerated tissue in a short time period.

The collagen may be uncrosslinked or partially or fully crosslinked using, for example, chemical crosslinking, UV radiation, dehydrothermal crosslinking, and combinations of these treatments. The crosslinking is carried out for a time and under conditions sufficient to provide a non-immunogenic collagen substrate layer 12. It is understood that the amount of crosslinking for a non-immunogenic collagen substrate layer 12 may be determined depending on the relation between the donor species and the recipient species. For example, a porcine derived collagen may be crosslinked from 80% to 100% to provide a non-immunogenic implant 10 in a non-pig recipient.

In embodiments where different substrate layer 12 materials are used, the rate of degradation and strength of the multilayer implant 10 may be tailored to the timing needs. For example, in an embodiment combining at least one synthetic polymer substrate layer 12 and at least one collagen substrate layer 12, the synthetic polymer substrate may resorb faster than the collagen substrate layer 12 and elicits a positive tissue response to make newly generated tissues develop into the collagen substrate layer 12.

The selection of substrate layers 12 may also enhance the healing process. For example, it may be desirable to incorporate layers of a slowly resorbing substrate with layers of a rapidly resorbing substrate. The presence of the slowly resorbing substrate may be used to enhance the strength of the microperforated implant because the rapidly resorbing substrate would initially elicit a tissue ingrowth response until it completely dissolved at which time the slowly resorbing substrate would continue to promote ingrowth. The slowly resorbing substrate may also provide enhanced strength to the multilayer implant 10 for a longer duration than a multilayer implant 10 containing several layers of a single resorbable substrate or layers of multiple resorbable substrates having the same resorption rates. For example, porcine substrate layers may be employed, each having different crosslinkage percentages to provide at least one different resorbability rate or a plurality of resorbability rates.

The microperforations 14 are less than about 10 micrometers in diameter. The microperforations 14 may also be less than about 1 micrometer, less than about 100 nanometers, or less than about 10 nanometers in diameter. The microperforations 14 may be of the same size within a substrate layer 12 or there may be different sizes within a single substrate layer 12 or between the substrate layers 12. The diameter of the microperforation 14 refers to the largest cross-section of the microperforation 14 substantially parallel to the substrate layer 12. For example, a circle, a square, an ellipse, or a non-regular shape microperforation 14 may be employed provided the largest cross-section is of an appropriate size. It may be desirable to provide pores of a sufficient diameter to fit material through the pores, for example red blood cells. The diameter of the microperforations 14 may be increased by displacing a part of the substrate layer 12 without removing the substrate material as depicted in FIG. 3. The overhang 20 on the bottom surface 18 is a result of enlarging the microperforation 14 diameter.

Returning to FIGS. 1 though 4, the substrate layers 12 have a punch density of about 1 to about 1,000 microperforation 14 punches per square inch. The microperforations 14 may be arranged in a pattern, or the microperforations 14 may be randomly placed throughout the substrate layer 12. The punch density of the multilayer implant 10 may be higher or lower than the provided ranges depending on the thickness of each substrate layer 12, the combination of substrate layers 12, and the desired rate of rehydration of the implant 10. In various embodiments, the implant 10 may include substrate layers 12 free from microperforations 14 paired with substrate layers 12 with microperforations 14. This arrangement may be useful with implants 10 made of a single substrate material, at least two different substrate materials, and when incorporating additional elements into the implant 10, as detailed later herein.

Dehydrated microperforated implants 10 may be rapidly rehydrated in less than about one hour or less than about 30 minutes. The microperforations 14 allow for a hydration fluid to quickly travel across a single substrate layer 12 or several substrate layers 12 and expedite hydration of the innermost substrate layers 12 or those layers located adjacent to at least two other substrate layers 12. The microperforated implants 10 may be arranged to even further expedite the rehydration. For example, arranging the substrate layers 12 such that at least one microperforation 14 on a substrate layer 12 is in fluid communication with at least one microperforation 14 on an immediately adjacent substrate layer 12 provides a channel or system of channels for efficient distribution of the hydration fluid. The fluid travels from the exposed edges (or perimeter) of the implant 10 to the top surface microperforation 16 of a substrate layer 12 and through the microperforation 14 to the bottom surface 18 of the substrate layer 12. When the fluid transverses the substrate layer 12, it wets or hydrates the adjacent substrate layer 12 top surface 16 or bottom surface 18 until it reaches a microperforation 14 in the adjacent substrate layer 12 and the process repeats until sufficient hydration of the multilayer implant 10 is achieved. Depending on the size of the overhang 20, displacement of the substrate layer 12 material may guide or funnel the hydration fluid to the surfaces 16, 18 of an adjacent substrate layer. For example, the microperforation 14 diameter and funneling may be selected to retain the hydration fluid against the innermost substrate layers 12 for a prolonged period of time.

Suitable hydration fluids may be aqueous, including but not limited to water, saline, and blood. Blood for hydration includes, but is not limited to, whole blood and blood components such as, red blood cells and components, white blood cells and components, plasma, plasma fractions, plasma serum, platelet concentrate, blood proteins, thrombin, and coagulation factors. A preferred hydration fluid is platelet concentrate.

The microperforated resorbable implant 10 may include additional elements such as autologous or allogeneic differentiated cells, autologous or allogeneic undifferentiated or stem cells and other biological agents, such as nutrient factors, growth factors, antimicrobials, anti-inflammatory agents, blood products, and mixtures thereof. These elements may be included between select substrate layers 12, between all substrate layers 12, or coated only on the outermost surface of the microperforated implant 10 or those top and/or bottom surfaces 16, 18 adjacent to only one other substrate layer 12. For example, in an embodiment where the additional elements are coated on the outermost layers, the elements may diffuse into the inner regions as the hydration media enters the microperforations 14. In other embodiments, the additional elements may be coated on the inner core substrate layers of the multilayer implant 10 or coated on alternating substrate layers 12 or the top surfaces 16 and/or bottom surfaces 18 of the substrate layers 12 of the microperforated implant 10.

Embodiments of the present invention also provide methods of preparing the microperforated implant 10. As depicted in FIG. 5, various methods generally include providing at least one substrate layer 12; perforating at least a region of the substrate layer 12; and dehydrating the substrate layer 12. Any of the operations may be performed in any order.

The microperforations 14 may be formed in at least a region of the substrate layer 12 by piercing miniscule holes in the substrate layer 12 with a needle. The needle may be an individual needle or a device with a plurality of needles such as those depicted in FIGS. 6 and 7. The rolling device 22 depicted in FIG. 6 may be rolled over the substrate layer 12 to provide the microperforations 14. The rolling device needles 24 may be of the same or different gauges and shapes. As depicted in FIG. 7, a plate device 26 may be used to create the microperforations 14. The plate needles 28 may be pressed into the substrate layer 12 to provide the microperforations. The rolling device 22 and the plate device 26 may be actuated by hand or automatically with a machine. The devices 22 and 26 may pierce select layers 12 individually or all of the layers of a multilayer implant simultaneously depending on the desired end product and preferred manufacturing techniques. The microperforations 14 may be enlarged by displacing the substrate without removing any additional material, by for example, stretching the microperforation 14 with a needle of the same or a larger diameter. Needles employed may be of any suitable gauge to provide the desired microperforation 14 size and punch density. The needle pierces the substrate layer 12 through the top surface 16 or the bottom surface 18. The needle may also “prick” only a single surface 16, 18 of the substrate layer 12 without engaging the opposing surface 16, 18, respectively.

The implant 10 has a minimal amount of the substrate material displaced to form the microperforations 14. Even though the microperforations 14 allow for the passage of a hydration fluid through the layers 12, the multilayer implant 10 has the same structural integrity and provides the same strength as a solid body implant without microperforations.

Returning to FIG. 5, the substrate layers 12 may be stacked. A precise and ordered stacking of the layers may place the microperforations 14 in fluid communication or the layers may be randomly stacked to achieve full, partial, or limited fluid communication between selected layers. For example, it may be desirable to arrange the microperforations 14 between the substrate layers 12 such that there is a pattern of angles between the microperforations 14 to facilitate hydrating fluid distribution. In various embodiments, it may be desirable to stack the substrate layers 12 such that the surface area of the multilayer implant 10 is the same as the surface area of any individual substrate layer 12. For example, a plurality of circular substrate layers 12 having the same diameter would stack to form a cylinder having a continuous radius and a plurality of square or rectangular substrate layers 12 would stack to form a block having a continuous cross-section. In other various embodiments, the implant may have a concave, convex, teardrop, or otherwise tapered shape, such as those described below, and there may be a surface area difference between the layers. In such embodiments, the greatest surface area of the implant 10 is not greater than the surface area of the layer 12 having the largest arc length or cross section length. The multilayer implant 10 is dehydrated using air drying, oven drying, vacuum drying, freeze drying, or any other suitable drying techniques.

In embodiments where the layers 12 are stacked prior to piercing and subsequently crosslinked together, the inherent porosity of the layers 12 is reduced by the crosslinking, thereby reducing the cumulative porosity of the implant 10. Accordingly, it may be advantageous to punch the microperforations 14 after stacking the layers 12 to form the implant 10.

The implant 10 may be treated to increase compatibility in the body. The implant may be sterilized using radiation, for example. Agents to increase ingrowth of tissues into the multilayer implant 10 may also be applied such as nutrient factors, growth factors, antimicrobials, anti-inflammatory agents, blood products, autologous or allogeneic differentiated cells, autologous or allogeneic undifferentiated or stem cells, and mixtures thereof.

Various embodiments of the present invention may be used to augment a site in need of soft tissue repair. The prepared and rehydrated multilayer implants 10 are placed at the site in need of soft tissue repair. Because of the rapid rehydration of the implant 10, shaping and preparation of the implant may be advantageously performed immediately prior to or during the soft tissue repair procedure. The microperforations 14 allow for quick diffusion of the hydration media through the multilayer implant 10 thereby providing flexibility in storage and use of the multilayer implant 10.

If needed, the implants 10 may be shaped prior to use. For example, the implant 10 may be shaped into a tear-drop, dome, or rounded shape to facilitate application of the implant 10 in rotator cuff repair procedures. The multilayer implant 10 may also be used to repair injuries to the acromioclavicular ligament, coracoclavicular ligament, or the coracoacromial ligaments in the shoulder may cause displacement of the clavicle. The implant 10 may be attached using any suitable attachment means such as sutures, screws, staples, etc. The methods may also be used in other regions of the body such as repair of a torn ulnar collateral ligament of the thumb, a torn biceps tendon, or in the knees, wrists, ankles, etc.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A multilayer microperforated implant comprising a plurality of microperforated substrate layers, wherein the microperforations have a diameter of less than about 10 micrometers.

2. The multilayer microperforated implant according to claim 1, wherein at least one substrate layer has a punch density of from about 1 to about 1,000 punches per square inch.

3. The multilayer microperforated implant according to claim 1, wherein the substrate layers comprise at least one resorbable material selected from the group consisting of polysaccharides, synthetic polymers, natural polymers, and mixtures thereof.

4. The multilayer microperforated implant according to claim 3, wherein the resorbable material is a polysaccharide selected from the group consisting of hyaluronic acid, chitin, chitosan, alginate, carboxymethylcellulose, and mixtures thereof.

5. The multilayer microperforated implant according to claim 3, wherein the resorbable material is a synthetic polymer selected from the group consisting of polymers and co-polymers of glycolic acid, L-lactic acid, D-lactic acid, urethane urea, trimethylene carbonate, dioxanone, caprolactone, hydroxybutyrate, orthoesters, orthocarbonates, aminocarbonates, and physical combinations thereof.

6. The multilayer microperforated implant according to claim 3, wherein the resorbable materials is a natural polymer selected from the group consisting of collagen, elastin, silk, fibrin, fibrinogen, and mixtures thereof.

7. The multilayer microperforated implant according to claim 6, wherein the natural polymer is collagen and the collagen is porcine derived.

8. The multilayer microperforated implant according to claim 1, wherein at least one substrate layer is a substantially non-resorbable collagen.

9. The multilayer microperforated implant according to claim 1, wherein the implant is dehydrated.

10. The multilayer microperforated implant according to claim 1, wherein at least one microperforation of each substrate layer is in fluid communication with at least one microperforation in an adjacent substrate layer.

11. The multilayer microperforated implant according to claim 1, wherein the microperforations are arranged to direct a hydration media to the inner most substrate layers.

12. The multilayer microperforated implant according to claim 1, wherein the implant is pre-fabricated.

13. The multilayer microperforated implant according to claim 12, wherein the pre-fabricated implant is a cartilage implant, a tendon implant, or a ligament implant.

14. The multilayer microperforated implant according to claim 1, wherein the implant comprises at least 8 layers.

15. A method of forming a microperforated implant comprising:
providing at least one substrate layer;
perforating at least a region of the substrate layer; and
dehydrating the substrate layer.

16. The method according to claim 15, wherein the perforating comprises contacting the substrate layer with a needle to displace less than about 10 micrometers of the material and form an opening.

17. The method according to claim 16, wherein the less than 10 micrometers opening is stretched to a greater diameter without removing additional material.

18. The method according to claim 15, wherein a plurality of substrate layers are stacked together to provide a multilayer microperforated implant.

19. The method according to claim 15, further comprising hydrating the microperforated implant with a hydration fluid such that upon contact with a hydration fluid, the hydration fluid transverses each of the substrate layers.

20. The method according to claim 15, wherein the hydration fluid is selected from the group consisting of water, saline, and blood selected from the group consisting of whole blood, platelet concentrate, and plasma.

21. A method of augmenting a site in need of repair, comprising:
a. providing dehydrated multilayer implant comprising a plurality of resorbable layers, wherein at least one resorbable layer includes perforations having a diameter of less than about 10 micrometers; and
b. placing the implant at a site in need of soft tissue repair.

22. The method according to claim 21, wherein each layer of the multilayer implant includes perforations having a diameter of less than about 10 micrometers.

23. The method according to claim 21, wherein the implant is hydrated with a hydration media selected from the group consisting of water, saline, and blood.
